(19) United States
(12) Patent Application Publication
(10) Pub. No.: US 2002/0022878 A1
(43) Pub. Date: Feb. 21, 2002

(54) Method for recellularization of a decellularized heart valve and heart valve produced thereby

(76) Inventor: E. Christopher Orton, Ft. Collins, CO (US)

Correspondence Address: Joseph E. Kovarik, SHERIDAN ROSS P.C., Suite 1200, 1560 Broadway, Denver, CO, 80202-5141, US

(21) Appl. No.: 09/909,914

(22) Filed: Jul. 20, 2001

Related U.S. Application Data
(60) Provisional application No. 60/219,548, filed on Jul. 20, 2000.

(51) Int. Cl. A61F 2/24

(52) U.S. Cl. 623/2.14; 623/918

(57) ABSTRACT

A method for producing a non-immunogenic and durable living graft involves the recellularization of a decellularized heart valve allograft or xenograft by recipient cells after implantation of such graft into a living patient. Decellularized allograft or xenograft grafts which have not been exposed to a cytotoxic environment are treated with either chemotactic factors or cell adhesion factors, or both, to retain desirable recipient cells into the tissue graft after implantation.

### FIELD OF THE INVENTION

The present invention is directed to a product and method relating to a living tissue graft that is both non-immunogenic and durable, and is more particularly directed to a method for producing a decellularized heart valve allograft or xenograft and recellularizing such graft by recipient cells after implantation.

### BACKGROUND OF THE INVENTION

Heart valve replacement is an established therapy for severe valvular insufficiency in humans. The ideal replacement heart valve would be hemodynamically efficient, nor-thrombogenic, and highly durable (i.e. several generations). Unfortunately, each of the currently available options for heart valve replacement have important drawbacks. Mechanical heart valves are hemodynamically efficient and highly durable, however they carry a substantial risk of valve thrombosis and require meticulous anticoagulation therapy with warfarin for the life of the patient. Glutaraldehyde-fixed tissue valves or bioprostheses (e.g. porcine valve xenografts) are less thrombogenic and do not require extended anticoagulation therapy after implantation, but have limited durability in humans lasting on average 5 to 10 years. Glutaraldehyde fixation of xenogenic grafts crosslinks the connective tissue component of the tissue and markedly decreases the antigenicity of the graft. The latter prevents a severe immune rejection that would destroy a fresh xenograft soon after implantation. However, at the same time, glutaraldehyde fixation kills the native cells of the graft and creates a permanent toxic environment in the connective tissue matrix that prevents any future recellularization of the graft. Thus, there can be no cellular component in a glutaraldehyde-fixed tissue graft that can maintain and repair the connective tissue matrix of the graft, and as a result the graft deteriorates over time.

Fresh and cryopreserved heart valve allografts do not require anticoagulation after implantation and their durability is superior to that of glutaraldehyde-fixed tissue valves. In theory, allografts have superior durability because they contain a living cellular component that can maintain the connective tissue matrix component of the graft. The current state of practice in human medicine is to undertake transplantation of heart valve allografts without any attempt at MHC matching or immunosuppressive drug therapy. The notion that heart valves are an “immunologically privileged site” and that they can be transplanted without eliciting tissue rejection has been challenged by several recent studies documenting humoral- and cell-mediated immune rejection of human heart valve allografts. It is widely suspected that this immune response contributes to deterioration of allografts over time. Another disadvantage of human heart valve allografts is that that they are limited by donor supply.

### SUMMARY OF THE INVENTION

In view of the above problems encountered with prior art grafts, one aspect of the present invention is the provision of a living graft that is both non-immunogenic and durable. The idea that allogenic or xenogenic tissue grafts that are composed principally of connective tissues (e.g. heart valves, pericardium, vessels, ligaments) can be rendered substantially non-immunogenic to a recipient by removal of native cells and cellular debris from the tissue graft is embodied by U.S. Pat. No. 4,801,299 (Klement et at.) and U.S. Pat. No. 4,776,853 (Brendel et al.), both of which are incorporated herein by this reference. In each of these patents it was recognized that the principal antigenic component of such tissue grafts would reside with its cellular and soluble protein component. Specifically, that treatment of a tissue graft with a series of hypotonic solutions, non-proteolytic enzymes (e.g. DNAse, RNAse), non-ionic and anionic detergents, and protease inhibitors could remove the cellular component of the graft and yet maintain the physical and mechanical properties inherent to the connective tissue matrix component of the graft. The result would be a tissue graft suitable for implantation that would have substantially decreased or no immunogenic reaction from the recipient toward the graft.

The present invention recognizes the key importance of recellularization of a decellularized heart valve allograft or xenograft by the recipient cells after implantation in order for the graft to be durable and non-thrombogenic. Specifically, the present invention involves treatment of a decellularized allograft or xenograft with substances to attract (i.e. chemotactic factors) and retain (i.e. cell adhesion factors) desirable recipient cells into the tissue graft after implantation. In the case of heart valve grafts, desirable cells may include fibroblasts, smooth muscle cells or myofibroblasts to maintain graft connective tissue matrix and thereby enhance graft durability, and endothelial cells to reduce thromobogenicity. It is also important that treatments aimed at decellularizing the tissue graft not result in a cytotoxic environment in the connective tissues that would prevent subsequent recellularization after implantation.

Specific chemotactic factors that may be used to treat a tissue graft prior to implantation that would attract fibroblasts and other desirable recipient cells into the tissue graft after implantation would include basic fibroblast growth factor (bFGF) or the glycosaminoglycan, heparan sulfate (heparin), or both. The chemoattractant properties of bFGF have been demonstrated both in vitro and in vivo. Basic FGF emulsified in collagen extract stimulates movement of fibroblasts into porous chambers implanted subcutaneously in rats. The strong affinity of bFGF for heparan sulfate protects it from degeneration and causes it to bind avidly to the glycosaminoglycan component of the connective tissue matrix. Thus, tissue grafts treated with bFGF with or without heparan sulfate prior to implantation retain these factors after treatment and establish a chemotactic gradient for fibroblasts and other desirable recipient cells after implantation. In one embodiment, treatment with heparan sulfate alone prior to implantation enhances attraction and retention of recipient cells within the graft after implantation. Cell adhesion factors such as fibronectin, which bind cells to connective tissue matrix components, can be used in retaining recipient cells within a tissue graft after implantation.

The use of bFGF to enhance ingrowth of fibroblasts into a decellularized tissue graft in vitro is embodied by U.S. Pat. Nos. 5,192,312, 5,772,695, 5,863,296, and 5,855617 (Orton) all of such patents incorporated herein by this reference. In one embodiment of the present invention, decellularized tissue allografts or xenografts are treated with bFGF prior to placing the grafts in co-culture with autogenous or allogenic cultured fibroblasts. The entire process is conducted in vitro, thus creating a cellularized hybrid tissue graft in vitro prior to implantation. Similarly the use of cell adhesion factors, such as fibronectin, and glycosaminoglycan, such as heparan sulfate, to enhance ingrowth and retention of fibroblasts into a decellularized tissue graft in vitro, is embodied by U.S. Pat. Nos. 5,613,982, 5,632,778, and 5,843,182 (Goldstein) all of which are incorporated herein by this reference.

In one embodiment of the present invention, recellularization of the graft is conducted subsequent to the step of implantation. In yet another embodiment of the invention, however, at least a portion of the graft is recellularized prior to implantation with the remaining recellularization occurring in vivo. Thus, the present invention relates to recellularization ex vivo in part and final recellularization of the remaining graft portions after implantation into a living patient. Further aspects of the present invention relate to the use of chemotactic factors and/or cell adhesion factors prior to implantation, subsequent to implantation, and/or a combination of both prior and subsequent to implantation to achieve the most durable graft. In one embodiment, at least one half of the graft is repopulated with cells prior to implantation with subsequent repopulation of the graft performed after implantation by administration of a suitable cell adhesion factors or chemotactic factors or growth factors, or a combination thereof.

One aspect of the present invention is to treat a decellularized tissue allograft or xenograft with chemotactic factors, such as bFGF or heparin sulfate, or cell adhesion factors such as fibronectin, or both, prior to implantation to enhance recellularization of the graft by autogenous recipient cells after implantation.

While various embodiments of the present invention have been described in detail, it will be apparent that further modifications and adaptations of the invention will occur to those skilled in the art. It is to be expressly understood that such modifications and adaptations are within the spirit and scope of the present invention.

What is claimed is:

1. A method for producing a heart valve allograft or xenograft having improved non-immunogicity and durability, comprising:
decellularizing a heart valve graft selected from the group consisting of an allograft and a xenograft;
implanting said graft into a living patient;
providing for recellularization of said graft subsequent to said step of implantation.

2. The method as set forth in claim 1, wherein said step of providing for recellularization comprises treating said graft with a factor selected from the group consisting of chemotactic factors and cell adhesion factors.

3. The method as set forth in claim 1, further comprising precluding contact between said graft and any fixative component prior to implantation of said graft.

4. The method as set forth in claim 3, wherein said fixation component comprises glutaraldehyde.

5. The method as set forth in claim 2, wherein said chemotactic factors are selected from the group consisting of bFGF, heparin sulfate and a glycosaminoglycan.

6. The method as set forth in claim 2, wherein said cell adhesion factors comprise fibronectin.

7. A heart valve graft produced by the method of claim 1.

8. The graft as set forth in claim 7, wherein said graft is not exposed to a cytotoxic environment that prevents subsequent recellularization of said graft after implantation.

9. The method of claim 1, further comprising contacting said graft with a cell selected from the group consisting of a fibroblast, a smooth muscle cell and a myofibroblast prior to said step of implantation.

10. A method for producing a tissue graft having improved non-immunogenicity and durability, comprising:
decellularizing a tissue graft selected from the group consisting of an allograft and a xenograft;
implanting said graft into a living patient; and
providing for recellularization of said graft subsequent to said step of implanting.
